(19) United States
(12) Patent Application Publication
(10) Pub. No.: US 2017/0251990 A1
(43) Pub. Date: Sep. 7, 2017

(54) NAVIGATION CARTS FOR A MEDICAL PROCEDURE

(71) Applicants: Leila KHERADPIR, Toronto (CA); William Wai-Leung LAU, Toronto (CA); Joshua Lee RICHMOND, Toronto (CA); Thanh Vinh VUONG, Toronto (CA)

(72) Inventors: Leila KHERADPIR, Toronto (CA); William Wai-Leung LAU, Toronto (CA); Joshua Lee RICHMOND, Toronto (CA); Thanh Vinh VUONG, Toronto (CA)

(21) Appl. No.: 15/518,295

(22) PCT Filed: Oct. 17, 2014
(86) PCT No.: PCT/CA2014/051005

§ 371 Date: Apr. 11, 2017

(51) Int. Cl. A61B 6/00; A61B 34/00; A61B 90/00; A61B 50/13

(52) U.S. Cl. CPC A61B 6/501; A61B 6/5247; A61B 50/13; A61B 34/25; A61B 90/37; B25J 18/025

(57) ABSTRACT

A cart for housing components of a medical navigation system is provided. The cart comprises a frame including a substantially horizontal base having a bottom side and a top side with wheels attached to the bottom side, a substantially vertical column attached to the top side of the base, and a ballast attached to the base to function as a counterweight to avoid tipping of the cart.

### TECHNICAL FIELD

The present disclosure is generally related to image guided medical procedures, and more specifically to navigation carts for a medical procedure.

### BACKGROUND

Conventional navigation carts for medical procedures create many hazards in the medical procedure room, including tripping hazards from cables, tipping hazards from equipment stands or towers, and general hazards associated with clutter because of too many equipment pieces being present in the room. Therefore, there is a need for an improved approach for providing navigation carts for use during a medical procedure.

### SUMMARY

One aspect of the present disclosure provides a cart for housing components of a medical navigation system. The cart comprises a frame including a substantially horizontal base having a bottom side and a top side with wheels attached to the bottom side, a substantially vertical column attached to the top side of the base, and a ballast attached to the base to function as a counterweight to avoid tipping of the cart. The cart may be modular in design and the components of the medical navigation system may be removably attached to the frame. The substantially vertical column is at least partially hollow therefore including a conduit for cables. The components of the medical navigation system may include a computing device attached to the frame, the computing device having a processor coupled to a memory and a wireless communication component for communicating wirelessly with a computing device associated with a second cart. The components of the medical navigation system may further include an uninterrupted power supply (UPS) attached to the base and coupled to the computing device for supplying uninterrupted power to the computing device. The cart may further include a robotic arm attached to an upper end of the substantially vertical column. The cart may further include a display of at least 55 inches in diagonal size mounted on the substantially vertical column, the ballast functioning to ensure stability of the card with the attached display. The cart may further include an arm having a first end and a second end, the first end attached to an upper end of the substantially vertical column, and a tracking camera attached to the second end of the arm such that the tracking camera is positionable above the display.

Another aspect of the present disclosure provides a medical navigation system including a first cart for housing components of the medical navigation system and a second cart for housing components of the medical navigation system. The first cart comprises a frame including a substantially horizontal base having a bottom side and a top side with wheels attached to the bottom side, a substantially vertical column attached to the top side of the base, and a ballast attached to the base to function as a counterweight to avoid tipping of the cart. The components of the medical navigation system include a computing device attached to the frame, the computing device having a processor coupled to a memory and a wireless communication component for communicating wirelessly with a computing device associated with the second cart. The second cart may be for housing further components of the medical navigation system. The second cart comprises a second frame including a second substantially horizontal base having a bottom side and a top side with wheels attached to the bottom side, a second substantially vertical column attached to the top side of the base, a second ballast attached to the base to function as a counterweight to avoid tipping of the cart; and a display of at least 55 inches in diagonal size mounted on the second substantially vertical column.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates the insertion of an access port into a human brain, for providing access to internal brain tissue during a medical procedure;

FIG. 2 shows an exemplary navigation system to support minimally invasive access port-based surgery;

FIG. 3 is a block diagram illustrating a control and processing system that may be used in the navigation system shown in FIG. 2;

FIGS. 4A is a flow chart illustrating a method involved in a surgical procedure using the navigation system of FIG. 2;

FIG. 4B is a flow chart illustrating a method of registering a patient for a surgical procedure as outlined in FIG. 4A;

FIG. 5 is a perspective view showing one example of a cart for housing components of a medical navigation system;

FIG. 6 is a perspective view showing another example of a cart for housing components of a medical navigation system;

FIG. 7A is a perspective view showing another example of a cart for housing components of a medical navigation system;

FIG. 7B is a front view of the cart shown in FIG. 7A;

FIG. 8A is a perspective view showing another example of a cart for housing components of a medical navigation system;

FIG. 8B is a side view of the cart shown in FIG. 8A;

FIG. 9 is a side view of the cart shown in FIG. 8 with additional components added; and

FIG. 10 is a side view of one variation of the cart shown in FIG. 8.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

Unless defined otherwise, all technical and scientific terms used herein are intended to have the same meaning as commonly understood by one of ordinary skill in the art. Unless otherwise indicated, such as through context, as used herein, the following terms are intended to have the following meanings:

As used herein, the phrase “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves.

As used herein the phrase “intraoperative” refers to an action, process, method, event or step that occurs or is carried out during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject or patient for imaging internal tissues, and methods of use thereof. Some embodiments of the present disclosure relate to minimally invasive medical procedures that are performed via an access port, whereby surgery, diagnostic imaging, therapy, or other medical procedures (e.g. minimally invasive medical procedures) are performed based on access to internal tissue through the access port.

The present disclosure is generally related to image guided medical procedures using a surgical instrument, such as a fibre optic scope, an optical coherence tomography (OCT) probe, a micro ultrasound transducer, an electronic sensor or stimulator, or an access port based surgery.

In the example of a port-based surgery, a surgeon or robotic surgical system may perform a surgical procedure involving tumor resection in which the residual tumor remaining after is minimized, while also minimizing the trauma to the intact white and grey matter of the brain. In such procedures, trauma may occur, for example, due to contact with the access port, stress to the brain matter, unintentional impact with surgical devices, and/or accidental resection of healthy tissue. A key to minimizing trauma is having the appropriate tools and equipment for use by a surgeon so that the medical procedure can be performed quickly, accurately, and safely.

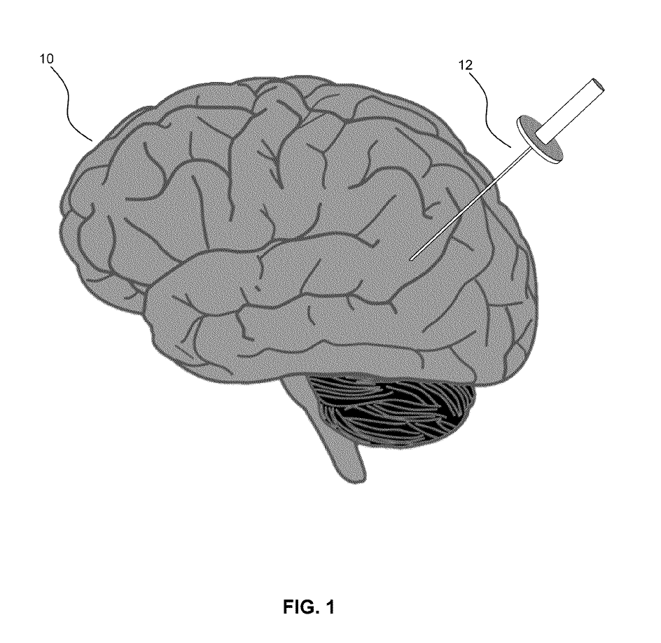

FIG. 1 illustrates the insertion of an access port into a human brain for providing access to internal brain tissue during a medical procedure. In FIG. 1, access port 12 is inserted into a human brain 10, providing access to internal brain tissue. Access port 12 may include such instruments as catheters, surgical probes, or cylindrical ports such as the NICO BrainPath. Surgical tools and instruments may then be inserted within the lumen of the access port in order to perform surgical, diagnostic or therapeutic procedures, such as resecting tumors as necessary. The present disclosure applies equally well to catheters, DBS needles, a biopsy procedure, and also to biopsies and/or catheters in other medical procedures performed on other parts of the body.

In the example of a port-based surgery, a straight or linear access port 12 is typically guided down a sulci path of the brain. Surgical instruments would then be inserted down the access port 12.

Optical tracking systems, used in the medical procedure, track the position of a part of the instrument that is within line-of-site of the optical tracking camera. These optical tracking systems also require a reference to the patient to know where the instrument is relative to the target (e.g., a tumor) of the medical procedure. These optical tracking systems require a knowledge of the dimensions of the instrument being tracked so that, for example, the optical tracking system knows the position in space of a tip of a medical instrument relative to the tracking markers being tracked. All of this requires the appropriate computer based equipment to be provided at the site of the medical procedure in a form that is easy for the surgical team to use and does not create any hazards in the room where the medical procedure will be performed.

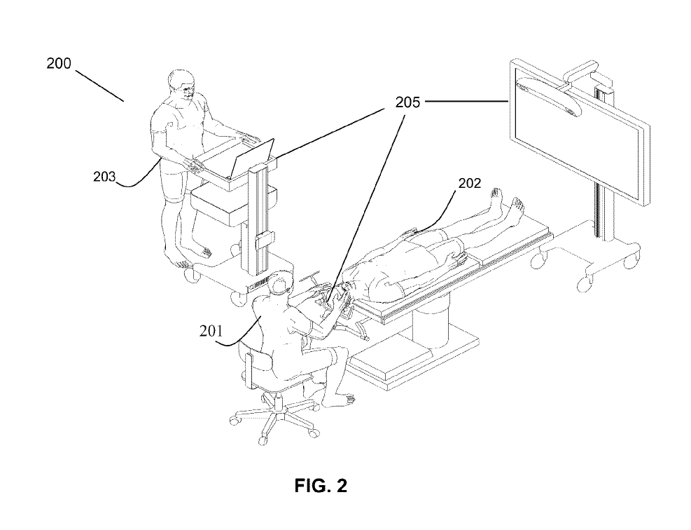

Referring to FIG. 2, an exemplary navigation system environment 200 is shown, which may be used to support navigated image-guided surgery. As shown in FIG. 2, surgeon 201 conducts a surgery on a patient 202 in an operating room (OR) environment. A medical navigation system 205 comprising an equipment tower, tracking system, displays and tracked instruments assist the surgeon 201 during his procedure. An operator 203 is also present to operate, control and provide assistance for the medical navigation system 205.

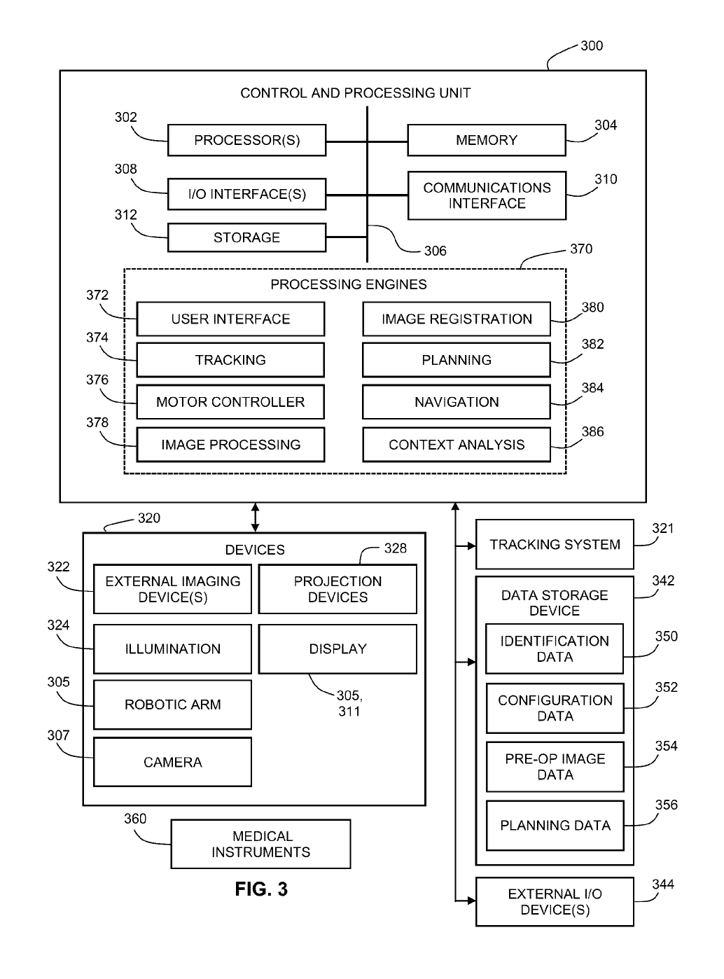

Referring to FIG. 3, a block diagram is shown illustrating a control and processing system 300 that may be used in the medical navigation system 200 shown in FIG. 3 (e.g., as part of the equipment tower). As shown in FIG. 3, in one example, control and processing system 300 may include one or more processors 302, a memory 304, a system bus 306, one or more input/output interfaces 308, a communications interface 310, and storage device 312. Control and processing system 300 may be interfaced with other external devices, such as tracking system 321, data storage 342, and external user input and output devices 344, which may include, for example, one or more of a display, keyboard, mouse, sensors attached to medical equipment, foot pedal, and microphone and speaker. Data storage 342 may be any suitable data storage device, such as a local or remote computing device (e.g. a computer, hard drive, digital media device, or server) having a database stored thereon. In the example shown in FIG. 3, data storage device 342 includes identification data 350 for identifying one or more medical instruments 360 and configuration data 352 that associates customized configuration parameters with one or more medical instruments 360. Data storage device 342 may also include preoperative image data 354 and/or medical procedure planning data 356. Although data storage device 342 is shown as a single device in FIG. 3, it will be understood that in other embodiments, data storage device 342 may be provided as multiple storage devices.

Medical instruments 360 are identifiable by control and processing unit 300. Medical instruments 360 may be connected to and controlled by control and processing unit 300, or medical instruments 360 may be operated or otherwise employed independent of control and processing unit 300. Tracking system 321 may be employed to track one or more of medical instruments 360 and spatially register the one or more tracked medical instruments to an intraoperative reference frame. For example, medical instruments 360 may include tracking markers such as tracking spheres that may be recognizable by a tracking camera 307. In one example, the tracking camera 307 may be an infrared (IR) tracking camera. In alternate embodiments, the tracking camera 307 may be an optical camera or a stereoscopic camera, capable of supporting 3D images or holograms. In another example, as sheath placed over a medical instrument 360 may be connected to and controlled by control and processing unit 300.

Control and processing unit 300 may also interface with a number of configurable devices, and may intraoperatively reconfigure one or more of such devices based on configuration parameters obtained from configuration data 352. Examples of devices 320, as shown in FIG. 3, include one or more external imaging devices 322, one or more illumination devices 324, a robotic arm, one or more projection devices 328, and one or more displays 205, 211.

Exemplary aspects of the disclosure can be implemented via processor(s) 302 and/or memory 304. For example, the functionalities described herein can be partially implemented via hardware logic in processor 302 and partially using the instructions stored in memory 304, as one or more processing modules or engines 370. Example processing modules include, but are not limited to, user interface engine 372, tracking module 374, motor controller 376, image processing engine 378, image registration engine 380, procedure planning engine 382, navigation engine 384, and context analysis module 386. While the example processing modules are shown separately in FIG. 3, in one example the processing modules 370 may be stored in the memory 304 and the processing modules may be collectively referred to as processing modules 370.

It is to be understood that the system is not intended to be limited to the components shown in FIG. 3. One or more components of the control and processing system 300 may be provided as an external component or device. In one example, navigation module 384 may be provided as an external navigation system that is integrated with control and processing system 300.

Some embodiments may be implemented using processor 302 without additional instructions stored in memory 304. Some embodiments may be implemented using the instructions stored in memory 304 for execution by one or more general purpose microprocessors. Thus, the disclosure is not limited to a specific configuration of hardware and/or software.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which, when executed by a data processing system, causes the system to perform various methods. The executable software and data may be stored in various places including for example ROM, volatile RAM, nonvolatile memory and/or cache. Portions of this software and/or data may be stored in any one of these storage devices.

Examples of computer-readable storage media include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions may be embodied in digital and analog communication links for electrical, optical, acoustical or other forms of propagated signals, such as carrier waves, infrared signals, digital signals, and the like. The storage medium may be the internet cloud, or a computer readable storage medium such as a disc.

At least some of the methods described herein are capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

According to one aspect of the present application, one purpose of the navigation system 205, which may include control and processing unit 300, is to provide tools to the neurosurgeon that will lead to the most informed, least damaging neurosurgical operations. In addition to removal of brain tumours and intracranial hemorrhages (ICH), the navigation system 205 can also be applied to a brain biopsy, a functional/deep-brain stimulation, a catheter/shunt placement procedure, open craniotomies, endonasal/skull-based/ENT, spine procedures, and other parts of the body such as breast biopsies, liver biopsies, etc. While several examples have been provided, aspects of the present disclosure may be applied to any suitable medical procedure.

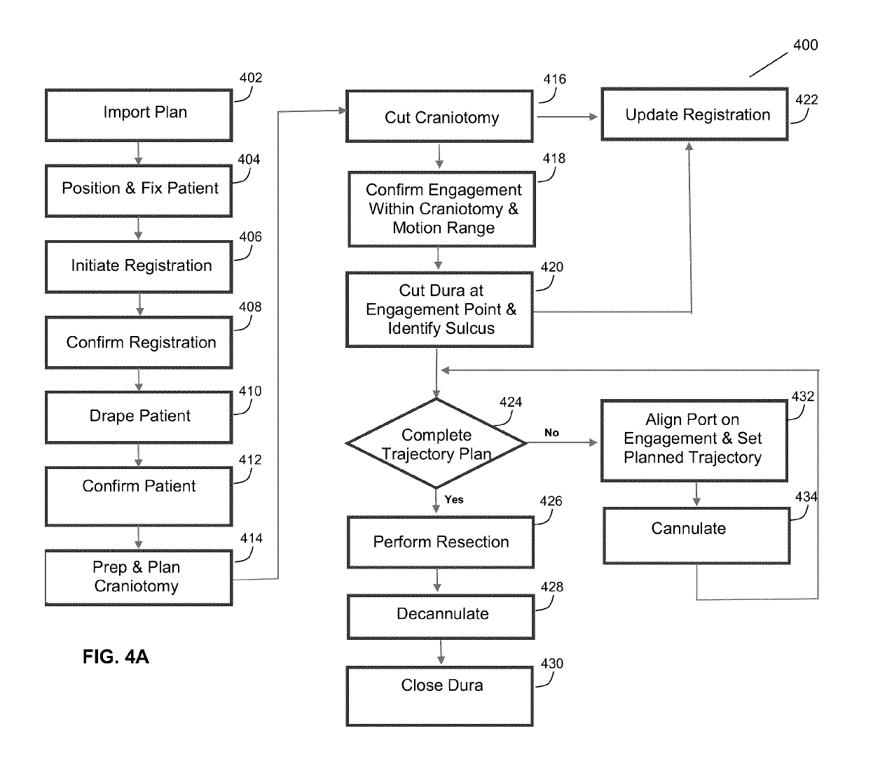

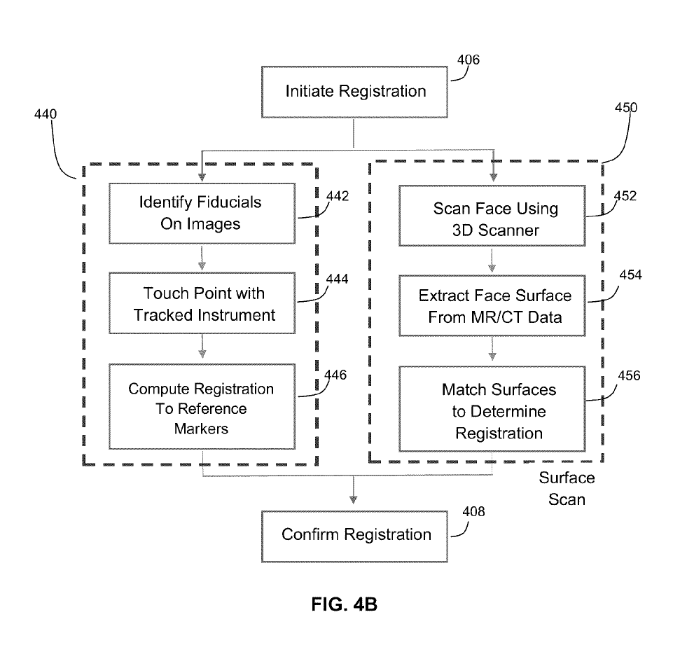

Referring to FIG. 4A, a flow chart is shown illustrating a method 400 of performing a port-based surgical procedure using a navigation system, such as the medical navigation system 200 described in relation to FIG. 2. At a first block 402, the port-based surgical plan is imported. A detailed description of the process to create and select a surgical plan is outlined in the international publication WO/2014/139024 “PLANNING, NAVIGATION AND SIMULATION SYSTEMS AND METHODS FOR MINIMALLY INVASIVE THERAPY”, which claims priority to U.S. Provisional Patent Application Ser. Nos. 61/800,155 and 61/924,993, which are both hereby incorporated by reference in their entirety.

Once the plan has been imported into the navigation system at the block 402, the patient is affixed into position using a body holding mechanism. The head position is also confirmed with the patient plan in the navigation system (block 404), which in one example may be implemented by the computer or controller forming part of the equipment tower 201.

Next, registration of the patient is initiated (block 406). The phrase “registration” or “image registration” refers to the process of transforming different sets of data into one coordinate system. Data may include multiple photographs, data from different sensors, times, depths, or viewpoints. The process of “registration” is used in the present application for medical imaging in which images from different imaging modalities are co-registered. Registration is used in order to be able to compare or integrate the data obtained from these different modalities.

Those skilled in the relevant arts will appreciate that there are numerous registration techniques available and one or more of the techniques may be applied to the present example. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT.

Referring now to FIG. 4B, a flow chart is shown illustrating a method involved in registration block 406 as outlined in FIG. 4A, in greater detail. If the use of fiducial touch points (440) is contemplated, the method involves first identifying fiducials on images (block 442), then touching the touch points with a tracked instrument (block 444). Next, the navigation system computes the registration to reference markers (block 446).

Alternately, registration can also be completed by conducting a surface scan procedure (block 450). The block 450 is presented to show an alternative approach, but may not typically be used when using a fiducial pointer. First, the face is scanned using a 3D scanner (block 452). Next, the face surface is extracted from MR/CT data (block 454). Finally, surfaces are matched to determine registration data points (block 456).

Upon completion of either the fiducial touch points (440) or surface scan (450) procedures, the data extracted is computed and used to confirm registration at block 408, shown in FIG. 4A.

Referring back to FIG. 4A, once registration is confirmed (block 408), the patient is draped (block 410). Typically, draping involves covering the patient and surrounding areas with a sterile barrier to create and maintain a sterile field during the surgical procedure. The purpose of draping is to eliminate the passage of microorganisms (e.g., bacteria) between non-sterile and sterile areas. At this point, conventional navigation systems require that the non-sterile patient reference is replaced with a sterile patient reference of identical geometry location and orientation. Numerous mechanical methods may be used to minimize the displacement of the new sterile patient reference relative to the non-sterile one that was used for registration but it is inevitable that some error will exist. This error directly translates into registration error between the surgical field and pre-surgical images. In fact, the further away points of interest are from the patient reference, the worse the error will be.

Upon completion of draping (block 410), the patient engagement points are confirmed (block 412) and then the craniotomy is prepared and planned (block 414).

Upon completion of the preparation and planning of the craniotomy (block 414), the craniotomy is cut and a bone flap is temporarily removed from the skull to access the brain (block 416). Registration data is updated with the navigation system at this point (block 422).

Next, the engagement within craniotomy and the motion range are confirmed (block 418). Next, the procedure advances to cutting the dura at the engagement points and identifying the sulcus (block 420).

Thereafter, the cannulation process is initiated (block 424). Cannulation involves inserting a port into the brain, typically along a sulci path as identified at 420, along a trajectory plan. Cannulation is typically an iterative process that involves repeating the steps of aligning the port on engagement and setting the planned trajectory (block 432) and then cannulating to the target depth (block 434) until the complete trajectory plan is executed (block 424).

Once cannulation is complete, the surgeon then performs resection (block 426) to remove part of the brain and/or tumor of interest. The surgeon then decannulates (block 428) by removing the port and any tracking instruments from the brain. Finally, the surgeon closes the dura and completes the craniotomy (block 430). Some aspects of FIG. 4A are specific to port-based surgery, such as portions of blocks 428, 420, and 434, but the appropriate portions of these blocks may be skipped or suitably modified when performing non-port based surgery.

When performing a surgical procedure using a medical navigation system 205, as outlined in connection with FIGS. 4A and 4B, the medical navigation system 205 must acquire and maintain a reference of the location of the tools in use as well as the patient in three dimensional (3D) space. In other words, during a navigated neurosurgery, there needs to be a tracked reference frame that is fixed relative to the patient's skull. During the registration phase of a navigated neurosurgery (e.g., the step 406 shown in FIGS. 4A and 4B), a transformation is calculated that maps the frame of reference of preoperative MRI or CT imagery to the physical space of the surgery, specifically the patient's head. This may be accomplished by the navigation system 205 tracking locations of fiducial markers fixed to the patient's head, relative to the static patient reference frame. The patient reference frame is typically rigidly attached to the head fixation device, such as a Mayfield clamp. Registration is typically performed before the sterile field has been established (e.g., the step 410 shown in FIG. 4A).

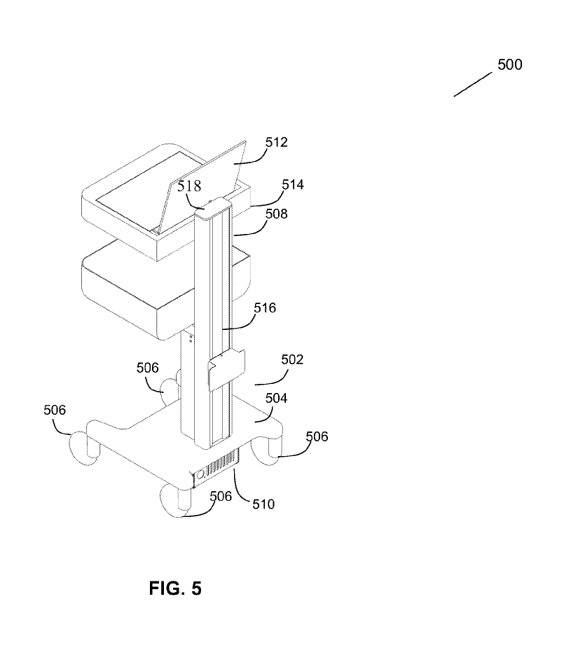

Referring to FIG. 5, a perspective view showing one example of a cart 500 for housing components of a medical navigation system is illustrated according to one aspect of the present application. The medical navigation system may be similar to the medical navigation system 205 (FIG. 2) that includes the control and processing system 300 (FIG. 3). The cart 500 includes a frame 502 that may have a base 504 having a bottom side and a top side. In one example, the base may be substantially horizontal and may have wheels 506 attached to the bottom side of the base. The frame 502 may further include a column 508 attached to the top side of the base. In one example, the column 508 may be substantially vertical. The frame 502 may further include a ballast 510 attached to the base, where the ballast may function as a counterweight to avoid tipping of the cart.

In one example, the ballast 510 may be attached to the bottom side of the base 504 between the wheels 506. The base 504 may have four corners and the four wheels 506 may be configured such that each wheel 506 is attached near a corner of the base 504. While the ballast 510 is shown attached to the bottom side of the base 504, the ballast 510 may also be attached to the top side of the base 504, or any other suitable location to meet the design criteria of a particular application. Likewise, while the example in FIG. 5 shows four wheels 506, three wheels, five wheels, six wheels, or any other suitable number of wheels may be used to meet the design criteria of a particular application, where an objective may include maximizing or achieving substantial stability of the cart 500.

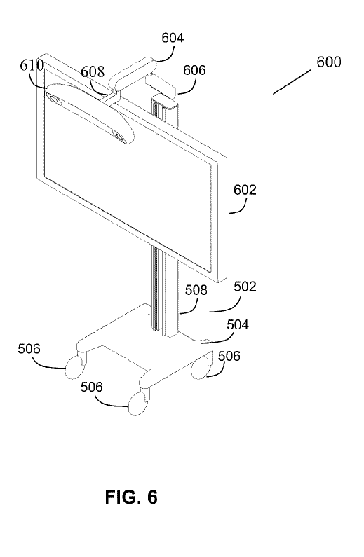

The components of the medical navigation system housed on the cart 500 may include a computing device 512 attached to the frame 502. In the example shown in FIG. 5, the computing device 512 includes a portable computer such as a laptop computer resting on a shelf 514 that is attached to the column 508. In one example, the shelf 514 may be foldable relative to the column 508, such as by a hinge mechanism attaching the shelf 514 to the column 508. The computing device 512 may also be considered part of the control and central processing unit 300 of navigation system 205. The computing device 512 may have a processor (e.g., processor 302) coupled to a memory (e.g., memory 304) and a wireless communication component (e.g., communications interface 310) for communicating wirelessly with a computing device associated with a second cart, such as the cart 600 discussed below in connection with FIG. 6. The wireless communications components 310 may include short and long range protocols such as Bluetooth, Zigbee, IRDA, Wi-Fi, GSM, CDMA, LTE or any other suitable existing or yet to be developed wireless communications protocol.

The components of the medical navigation system may further include an uninterrupted power supply (UPS) attached to the base 504 and coupled to the computing device 512 for supplying uninterrupted power to the computing device 512. In the example shown in FIG. 5, the UPS may be located in the same position as the ballast 510. Since a UPS is typically heavy, the UPS may form part of the ballast 510 or may be located next to the ballast 510 to help stabilize the cart 500. In another example, the UPS may be located on top of the base 504. While some examples for the location of the UPS have been provided, the UPS may be located in any suitable location on the cart 500 to meet the design criteria of a particular application.

In one example, the column 508 may be at least partially hollow therefore including a conduit for cables and power management. Column 508 may be used for cable management for concealing cables (e.g., power cord, or networking, USB, audio, video cables) connecting computing device 512 to a UPS, external hard drives, CDROMs, or wireless communications component 310 on navigation cart 500 and to other components of navigation system 205 such as display 602 and/or tracking camera 610 on auxiliary cart 600, as seen in FIG. 6. The column 508 may be a fixed length or may also be extendible, such as being a modular design where smaller subsections of the column 508 may be attached together to create a needed length of the column 508. Vertical extension of column 508 may be raised (and also lowered) by a release mechanism either manually or automatically (i.e., motorized). The hollow conduit of column 508 may also house a power bar or electrical extension cord.

In FIG. 5, a back cover 516 is shown attached to the column 508 to retain and hide cables placed within the column 508. An end cap 518 is also shown placed on the top end of the column 508. In the situation where a longer column is desired, the end cap 518 may be removed and additional sections of the column 508 may be attached to the top end of the column 508. End cap 518 may be removed and replaced by a connecting joint (not shown) that connects the top of column 508 to a robotic arm, tracking camera, imaging camera or other accessories used by the navigation system 205.

Referring now to FIG. 6, a perspective view showing another example of an auxiliary cart 600 is illustrated for housing additional components of a medical navigation system 205. In one example, auxiliary cart 600 may be similar to cart 500 since the carts 500 and 600 are designed to be modular and share many of the same components. In this regard, like components are shown with like reference numerals, where auxiliary cart 600 also includes the frame 502, the base 504, the wheels 506, the column 508, and the ballast 510 (not visible in FIG. 6). Auxiliary cart 600 may have a column 508 that is taller than cart 500, which may be constructed using two or more like columnar portions attached together. However, in another example columnar portions may be available in different lengths and column 508 of cart 600 may be comprised of only one columnar portion. In the example shown in FIG. 6, auxiliary cart 600 also include a display 602 mounted on the column 508. In one example, the display 602 may be at least 55 inches in diagonal size, therefore providing a superior viewing experience for the surgeon as opposed to conventional solutions. In one example, the display 602 is mounted on a front side of the column 508. The ballast 510 may be designed to ensure stability of the card with the attached display 602, for example by ensuring that ballast 510 is heavy enough to prevent tipping of auxiliary cart 600 with the display 602 attached.

The auxiliary cart 600 may also include an arm 604 having a first end 606 and a second end 608, where the first end 606 is attached to an upper end of the column 508 and the second end 608 is attached to a tracking camera 610 (e.g., the camera 307 and/or the tracking system 321) such that the tracking camera 610 is positionable above the display 602, as shown in FIG. 6.

The components of the medical navigation system housed on the auxiliary cart 600 may include a computing device 512 (not shown in FIG. 6) attached to the frame 502. In the example shown in FIG. 6, the computing device 512 may be integrated into the display 602 or attached behind the display 602. The computing device 512 may have a processor (e.g., processor 302) coupled to a memory (e.g., memory 304) and a wireless communication component (e.g., communications interface 310) for communicating wirelessly with a computing device associated with the first cart 500. The wireless communications components 310 may include Bluetooth, Zigbee, IRDA, Wi-Fi, GSM, CDMA, LTE or any other suitable existing or yet to be developed wireless communications protocol. In this way, the display 602 and/or tracking camera 610 may communicate wirelessly with the computing device 512 of FIG. 5 such that no cables need to be placed across the operating room floor, thereby eliminating a tripping hazard.

The components of the medical navigation system included in FIG. 6 may further include an uninterrupted power supply (UPS) attached to the base 504 and coupled to the display 602 for supplying uninterrupted power to the display 602. In the example shown in FIG. 6, the UPS may be located in the same position as the ballast 510 (e.g., on the bottom of the base 504).

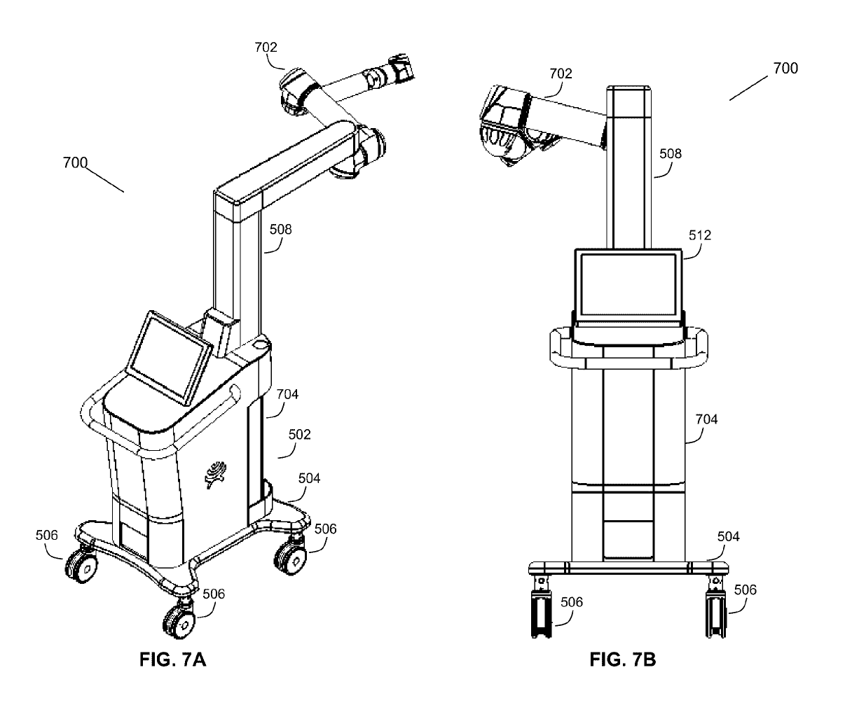

Referring to FIG. 7A, a perspective view of another example of a cart 700 is shown for housing components of a medical navigation system. FIG. 7B is a front view of the cart shown in FIG. 7A and FIGS. 7A and 7B are discussed concurrently and collectively referred to as FIG. 7. In one example, cart 700 may be similar to cart 500 since the carts 500 and 700 are designed to be modular and share many of the same components. In this regard, like components are shown with like reference numerals, where cart 700 also includes the frame 502, the base 504, the wheels 506, the column 508, the ballast 510 (not visible in FIG. 7), and the computing device 512. In the example shown in FIG. 7, the ballast 510 may be placed on the top side of the base 504. Cart 700 may have a column 508 that is taller than cart 500, which may be constructed using two or more like columnar portions attached together. However, in another example columnar portions may be available in different lengths and column 508 of cart 700 may be only one columnar portion.

The cart 700 may include a robotic arm 702 attached to an upper end of the column 508. The end effector of the robotic arm 702 may be attached to an optical camera or a surgical microscope (not shown), providing enhanced images (e.g., picture or video) of the surgical procedure, which may be displayed on display 602. Optionally, the end effector of robotic arm 702 may also be equipped with alternate imaging modality devices (i.e., MRI probe, Raman spectroscopy probe, ultrasound probe) which may provide intraoperative multi-modal reading of information for the surgical procedure.

The cart 700 may further include a removable cabinet 704 resting on the top side of the base 504. The cabinet 704 may house a computing device. In one example, a portable computing device such as a laptop computer may rest on top of the cabinet 704. In another example, the cabinet 704 may have a portable computing device or a desktop computing device inside the cabinet 704, with a monitor, keyboard, mouse or other I/O devices accessible either on top of the cabinet 704 or on a front side of the cabinet 704. The cart 700 may further include an integrated memory storage device for connection to a computing the device. In one example, the integrated memory storage device may include an optical drive, an external hard disk drive, a CD drive, a DVD drive, or a Bluray drive, or any combination thereof. The integrated memory storage device may be built into the cabinet 704 and may be connected to the computing device using any suitable interface, such as a USB interface cable.

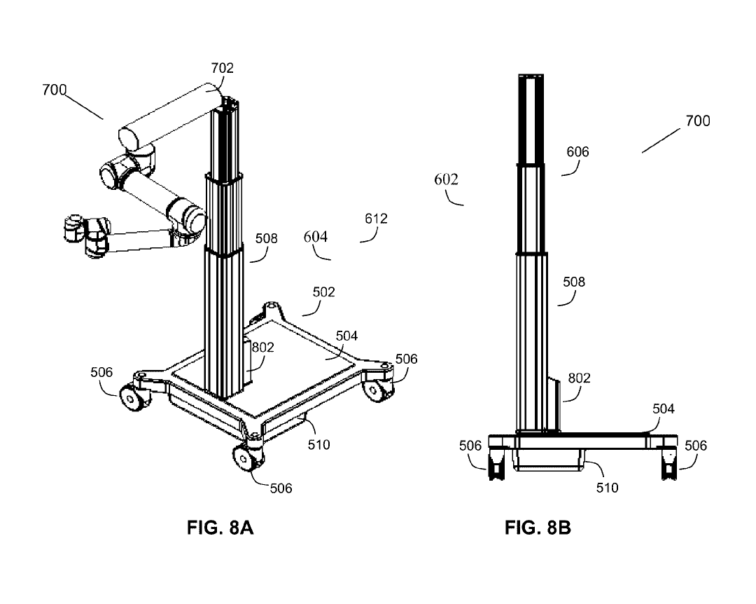

Referring now to FIG. 8A, a perspective view is shown illustrating another example of the cart 700 with removable cabinet 704 removed. FIG. 8B is a side view of the cart 700 shown in FIG. 8A and FIGS. 8A and 8B are discussed concurrently and collectively referred to as FIG. 8. FIG. 8 shows cart 700 including the frame 502, the base 504, the wheels 506, the column 508, the ballast 510, and the robotic arm 702. In one example, the column 508 shown in FIG. 8 may be telescoping having electromechanical actuators (not shown) for lowering and raising the robotic arm 702. The electromechanical actuators may be connected to a control system that can be connected to a computing device using a suitable interface, such as a USB cable. In another example, the column 508 is modular and is at least partially hollow comprised of more than one columnar portion, such as three portions shown in FIG. 8. The at least partially hollow column functions as a wire conduit for computing devices located in or on the cart 700. The cart 700 further has a UPS 802. The UPS 802 may be located on the top side of the base 504 adjacent the column 508 and the UPS 802 may be coupled to the computing device 512 for supplying uninterrupted power to the computing device 512. Since the UPS is typically heavy, it may also aid the ballast 510 to stabilize the cart 700. While the example shown in FIG. 8 shows the ballast 510 on the bottom side of the base 504 and the UPS 802 on the top side of the base 504, both the UPS 802 and the ballast 510 may be located in either location either separately or together, depending on the design criteria of a particular application.

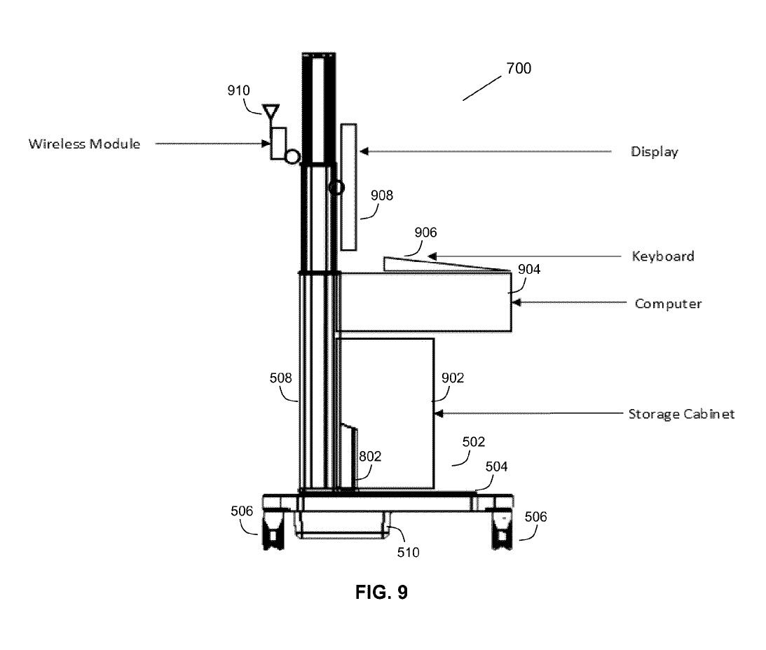

Referring now to FIG. 9, a side view of the cart 700 shown in FIG. 8 is illustrated with additional components added in a modular fashion. FIG. 9 shows cart 700 including the frame 502, the base 504, the wheels 506, the column 508, the ballast 510, and the UPS 802. The cart 700 may further include a storage cabinet 902 resting on top of the top side of the base 504. In one example, the storage cabinet 902 may be removably attached to the cart 700. The cart 700 may further include a computer 904 attached to the column 508. The computer 904 may be either directly attachable to the column 508 or may be housed in a cabinet attached to the column 508. The computer 904 may have a keyboard 906 or input devices resting on top of the computer 904. The cart 700 may further have a display 908 attached to the column 508 and a wireless communications module 910 attached to the column 508. The UPS 802, computer 904, keyboard 906, display 908, and wireless communications module 910 may all be interconnected or coupled by wires that may use the conduit inside the column 508, as appropriate, for hiding the wires.

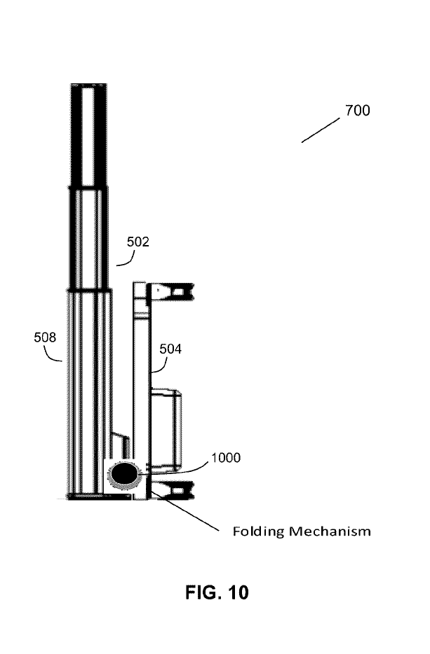

FIG. 10 is a side view of one variation of the cart 700 shown in FIG. 8. In the example shown in FIG. 10, the frame 502 may further include a hinge 1000 attaching the column 508 to the base 504 such that the column 508 is foldable relative to the base 504. While a hinge has been used as an example of a mechanism to facilitate folding, any suitable mechanism may be used to meet the design criteria of a particular application.

In yet another example, the carts 500 and 600 may be provided together as a single solution. A medical navigation system may include a first cart 500 for housing components of the medical navigation system. The first cart 500 comprises a frame including a substantially horizontal base 504 having a bottom side and a top side with wheels 506 attached to the bottom side, a substantially vertical column 508 attached to the top side of the base 504, and a ballast 510 attached to the base 504 to function as a counterweight to avoid tipping of the cart 500. The components of the medical navigation system include a computing device 512 attached to the frame 502, the computing device 512 having a processor coupled to a memory and a wireless communication component for communicating wirelessly with a computing device associated with a second cart 600. The second cart 600 may be for housing further components of the medical navigation system. The second cart 600 comprises a second frame 502 including a second substantially horizontal base 504 having a bottom side and a top side with wheels 506 attached to the bottom side, a second substantially vertical column 508 attached to the top side of the base, a second ballast 510 attached to the base 504 to function as a counterweight to avoid tipping of the cart 600, and a display 602 of at least 55 inches in diagonal size mounted on the second substantially vertical column 508.

In one example, the cart 500 may be referred to as a navigation cart and the auxiliary cart 600 may be referred to as an auxiliary cart. The two separate carts 500 and 600 allow the surgeon to position the cart 600 with the tracking camera 610 and monitor 602 in the optimal position with respect to his patient and site of operation. The navigation cart 500 may hold the computing device 512 and may have an operator and may or may not need to be close to the auxiliary cart 600. Therefore, the cart 500 may be positioned as far as 30 feet away from the auxiliary cart 600 and away from other instruments to reduce clutter in an operating room. The computing device 512 may be laptop computer that is easy to upgrade. The computing device 512 may sit on top of the shelf 514 and switching computing devices 512 may be a simple as switching cables. The carts 500, 600, and 700 may be configured where each main component is modular and may be easily removed and upgradeable.

Any of the carts 500, 600, and 700 may include a computing device such as a laptop, keyboard and mouse, an isolation transformer, an NDI USB Hub for Polaris Spectra, power adaptors for the laptop and Polaris camera, an optical drive and USB port, a Polaris Spectra tracking camera, an NDS 55″ G2 Radiance medical grade monitor, Polaris Spectra adaptor and handle to the arm, and/or a storage drawer. Enclosures such as the enclosure 704 may be designed to accommodate one or more of an isolation transformer, an NDI USB hub, a DVD, Blu-Ray or other Optical drive, a USB drive, a patient reference adaptor and extension arm to Mayfield clamp, provide for cable strain relief, and/or port panel, and/or a power adaptor enclosure. Computing device may also include a desktop personal computer (PC), a tablet, smartphone and/or an embedded computing device.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

We claim:

1. A cart for housing components of a medical navigation system, the cart comprising:
a frame including:
a substantially horizontal base having a bottom side and a top side with wheels attached to the bottom side;
a substantially vertical column attached to the top side of the base; and
a ballast attached to the base to function as a counterweight to avoid tipping of the cart;
wherein the cart is modular in design and the components of the medical navigation system are removably attached to the frame and wherein the substantially vertical column is at least partially hollow therefore including a conduit for cables.

2. The cart according to claim 1, wherein the components of the medical navigation system include a computing device, the computing device having a processor coupled to a memory and a wireless communication component for communicating wirelessly with a computing device associated with a second cart.

3. The cart according to claim 2, wherein the components of the medical navigation system further include an uninterrupted power supply (UPS) attached to the base and coupled to the computing device for supplying uninterrupted power to the computing device.

4. The cart according to claim 1, wherein the cart further includes a robotic arm attached to an upper end of the substantially vertical column.

5. The cart according to claim 1, wherein the cart further includes a display mounted on the substantially vertical column, the ballast functioning to ensure stability of the card with the attached display.

6. The cart according to claim 5, wherein the display is of at least 55 inches in diagonal size.

7. The cart according to claim 5, the cart further including:
an arm having a first end and a second end, the first end attached to an upper end of the substantially vertical column, and
a tracking camera attached to the second end of the arm such that the tracking camera is positionable above the display.

8. The cart according to anyone of claims 1-7, wherein the substantially vertical column is extendible.

9. The cart according to anyone of claims 1-8, wherein the frame further includes a hinge attaching the substantially vertical column to the substantially horizontal base such that the column is foldable relative to the base.

10. The cart according to anyone of claims 1-9, wherein the wheels are lockable.

11. The cart according to anyone of claims 1-10, wherein the cart further includes a foldable shelf attached to the substantially vertical column for supporting a portable computing device.

12. The cart according to anyone of claims 1-11, wherein the cart further includes a removable cabinet resting on the top side of the substantially horizontal base, the cabinet for housing a computer.

13. The cart according to anyone of claims 1-12, wherein the ballast is attached to the bottom side of the base between the wheels, the base having four corners and four wheels, each wheel being attached near a corner of the base.

14. The cart according to anyone of claims 1-13, wherein the cart further includes an integrated memory storage device for connection to a computing the device, the integrated memory storage device including at least one of an optical drive, an external hard disk drive, a CD drive, a DVD drive, and a Blu-ray drive.

15. A medical navigation system comprising:
a first cart for housing components of the medical navigation system, the first cart comprising:
a frame including:
a substantially horizontal base having a bottom side and a top side with wheels attached to the bottom side;
a substantially vertical column attached to the top side of the base; and
a ballast attached to the base to function as a counterweight to avoid tipping of the cart,
the components of the medical navigation system including:
a computing device attached to the frame, the computing device having a processor coupled to a memory and a wireless communication component for communicating wirelessly with a computing device associated with a second cart;
the second cart for housing further components of the medical navigation system, the second cart comprising:
a second frame including:
a second substantially horizontal base having a bottom side and a top side with wheels attached to the bottom side;
a second substantially vertical column attached to the top side of the second base;
a second ballast attached to the second base to function as a counterweight to avoid tipping of the second cart; and
a display mounted on the second substantially vertical column.

16. The medical navigation system according to claim 15, wherein the components of the medical navigation system further include an uninterrupted power supply (UPS) attached to each of the first base and the second base and coupled to, respectively, the computing device for supplying uninterrupted power to the computing device and the display for supplying uninterrupted power to the display.

17. The medical navigation system according to any one of claims 15-16, wherein the first cart further includes a robotic arm attached to an upper end of the first substantially vertical column.

18. The medical navigation system according to any one of claims 15-17, the second cart further including:
an arm having a first end and a second end, the first end attached to an upper end of the second substantially vertical column, and
a tracking camera attached to the second end of the arm such that the tracking camera is positionable above the display.

19. The medical navigation system according to anyone of claims 15-18, wherein the substantially vertical column is at least partially hollow therefore including a conduit for cables, the substantially vertical column being extendible.

20. The medical navigation system according to any one of claims 15-19, wherein the display is of at least 55 inches in diagonal size.
